(19) United States
(12) Patent Application Publication
(10) Pub. No.: US 2007/0060865 A1
(43) Pub. Date: Mar. 15, 2007

(54) Device for the targeted, controllable delivery or drawing of a liquid or viscous substance

(76) Inventor: Max Wyssmann, Herzogenbuchsee (CH)

Correspondence Address: BARNES & THORNBURG LLP, 750-17TH STREET NW, SUITE 900, WASHINGTON, DC, 20006-4675, US

(21) Appl. No.: 10/596,637

(22) PCT Filed: Dec. 17, 2004
(86) PCT No.: PCT/EP04/14384

§ 371 Date: Jun. 19, 2006

(30) Foreign Application Priority Data
Dec. 20, 2003 (DE) 103 60 185.6

(51) Int. Cl. A61M 3/00

(52) U.S. Cl. 604/24

(57) ABSTRACT

A device for the targeted, controllable deliver or drawing of a liquid or viscous substance, comprises a reservoir and a plunger that subdivides the reservoir into a storage space for the viscous substance and a pressure space for gas. The storage space for the viscous substance leads to a discharge opening for the viscous substance, and an insert is placed in the pressure space inside the reservoir and contains at least one gas generator cell and a circuit for controlling the running time. The wall of the reservoir is, at least in sections, multilayered and translucent, whereby at least two layers are made of different chemical substances.

The invention relates to a device for the targeted, controllable delivery or drawing of a liquid or viscous substance according to the preamble of claim 1.

A device of this type is known from European Patent Document EP 0 598 867 B1 (Wyssmann). The delivery of small, particularly comparatively valuable liquid or viscous substances according to a previously defined time schedule, which has to be precisely observed, by means of an automatic autonomous control of the program flow—essentially without any intervention from the outside—is carried out particularly by means of devices known in principle from the above-mentioned European Patent Document EP 0 598 867 B1 of the same type.

Further state of the art is illustrated in German Patent Documents DE 195 22 451 A1, DE 35 32 335 A1, DE 92 02 740 U1, European Patent Documents 05 98 867 B1, EP 0 362 328 B1 (two-layer construction, transparent plastic material with a gastight metal coating which, however, is not transparent) and European Patent Document EP 0343 157 B1.

Suitable gas generating cells for such devices are described, for example, in German Patent Document DE 35 32 335 A1 (Winsel) or European Patent Document EP 0 343 157 B1 (Winsel). A corresponding device which can be used for the injection or infusion of a pharmaceutical solution is described in European Patent Document EP 0 278 954 B1 (Winsel).

The devices of the above-mentioned type have been successful per se. They also exist as suction devices (sample collecting devices, etc.).

However, the losses, which occur particularly at higher counterpressures and/or temperatures and/or longer running times and cannot be calculated in a sufficiently precise manner, present problems because of the diffusion of the gas generated by the gas generating cell for driving the piston for the discharge of the liquid or viscous substance from the reservoir.

It is an object of the invention to solve this problem.

The invention solves this task by means of the object of claim 1.

Advantageous embodiments are contained in the subclaims.

It is particularly advantageous for the wall of the reservoir to be constructed in three layers at least in sections. It preferably consists of transparent, translucent layers such that the filling level always remains visible. In contrast to a barrier layer made of metal, the entire cylinder jacket can therefore have a translucent construction.

In this case, it is useful for the inner and outer layer of the three-layer construction to consist of a preferably transparent plastic material, the center layer between the two preferably transparent plastic layers consisting of a material, particularly a solid material or a liquid which is also preferably transparent and has a low diffusion coefficient for the gas generated by the gas generating cell and delivered into the pressure chamber.

Particularly preferably, the outer and inner layers consist of transparent PET. Also preferably, the center barrier layer consists particularly of a transparent polyamide, which can even be processed as a solid layer and preferably has a thickness of 30-60%, particularly 40-50%, particularly preferably 45% of the entire wall. Particularly good results are achieved in this manner, and a particularly good compromise is implemented between a good sealing and a stable design. As an alternative, EVOH can also be used as a barrier layer.

Such an arrangement can be produced by a modern plastic injection process (co-injection or multi-material molding). It is expedient, for example, to use a gas barrier material, such as EVOH.

A special field of application involves lubricant input devices, in the case of which the gas generated by the gas generating cell and used for advancing the piston is hydrogen. The material is also useful for implementing corresponding suction devices.

In the following, the invention will be described in detail by means of an embodiment with reference to the drawing.

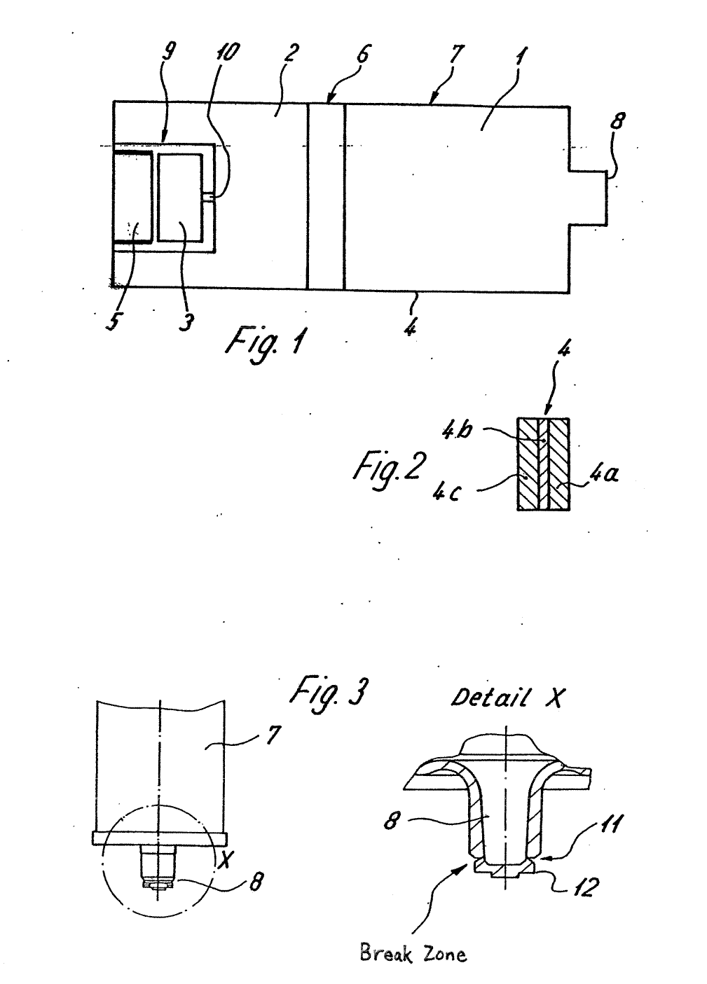

FIG. 1 is a schematic view of a device according to the invention;

FIG. 2 is an enlarged representation of the housing wall of the device from FIG. 1;

FIG. 3 is a view of a detail of another device.

FIG. 1 illustrates a preferably cylindrical reservoir 7 in which a piston 6 is displaceably guided.

The piston 6 divides the reservoir 7 into a pressure chamber 2 as well as a storage chamber 1 for viscous substances, particularly lubricants.

At one of its axial ends—on one side of the piston 6 on which the lubricant 1 is received in the storage chamber 1—the reservoir 7 has an discharge opening 8 and, at its opposite axial end, which is oriented toward the pressure chamber 2, it has an insert 9 in which at least one gas generating cell 3 as well as an electric circuit 5 and a device for controlling the running time (rotary switch or the like), which is not visible here, are accommodated.

At least one passage opening 10 between the gas generating cell 3 and the pressure chamber 2 permits the passage of gas, which is supplied by the gas generating cell, into the pressure chamber 2. Depending on the adjustment of the running time control—see the above-mentioned state of the art in this respect—the generating of gas, particularly hydrogen, now takes place, so that the piston 6 is moved by the gas generating cell and pushes the lubricant 1 out of the discharge opening 8.

As illustrated in FIG. 2, the wall 4 of the reservoir 7 has a multi-layer construction at least in sections, but preferably completely. In this case, it is useful to select a three-layer construction with inner and outer layers 4a, 4c produced of a transparent plastic material and, to construct a barrier layer 4b between these, particularly of a liquid material permitting only a very slight diffusion of gas, particularly hydrogen.

In this manner, not only the loss of gas, particularly of hydrogen at a high counterpresssure at the discharge opening, is clearly reduced, but the precision of the delivery of the viscous substance from the device is also increased, particularly at higher pressures or longer delivery times. Thus, it is, for example, conceivable to precisely deliver lubricants by means of the device according to the invention also for time periods of up to one year or more and to still satisfactorily operate the device even at counterpressures of over 5 bar. An analogous situation applies to increased temperatures which may also result in a higher pressure.

According to an embodiment of the invention, a closing device 12, which can be detached, particularly broken off by way of a predetermined breaking point, is molded onto the discharge opening 8.

This idea improves the removal of the molded-on closing pin at the funnel to the lubricant output device.

Function: The closing pin can be twisted off by means of a standard tool (key, pliers). This causes no brows projecting into the pipe cross-section as a result of the specific shaping and the rotating movement.

### REFERENCE NUMBERS

Lubricant 1
Pressure chamber 2
Gas generating cell 3
Wall 4
Electronic circuit 5
Piston 6
Reservoir 7
Outer layer 7a, 7c
Barrier layer 7b
Discharge opening 8
Insert 9
Passage opening 10
Predetermined breaking point 11
Closing device 12

1. A device for the targeted, controllable delivery or drawing of a liquid or viscous substance, comprising:
a) a reservoir having a piston dividing the reservoir into a storage chamber for the viscous substance and a pressure chamber for gas;
b) the storage chamber for the viscous substance leading into a discharge opening in the reservoir for the viscous substance;
c) an insert in the pressure chamber, which insert contains at least one gas generating cell and a circuit for the running-time control; and
d) the wall of the reservoir being constructed in several layers, at least two of the layers consisting of different chemical substances, at least one of the layers, which form the wall of the reservoir, having a lower diffusion coefficient for the gas to be generated by the gas generating cell than the other layer(s), and the wall of the reservoir consisting of one of transparent and translucent layers.

2. A device, according to claim 1, the inner and the outer layer of a three-layer wall consist of one of a transparent and translucent plastic material, the center layer between the two transparent layers consisting of transparent material which has a lower diffusion coefficient for the gas to be generated by the gas generating cell than the inner and the outer layer.

3. A device, according to claim 2, wherein the center layer consists of one of a solid material and of a liquid which is transparent and has a lower diffusion coefficient for the gas to be generated by the gas generating cell than the inner and the outer layer.

4. A device according to claim 1, including a closing device, which can be detached, is molded to the discharge opening.

5. A device according to claim 2, wherein the outer and inner layers consist of transparent PET.

6. A device according to claim 2, wherein the center barrier layer consists of polyamide.

7. A device, according to claim 2, wherein the center barrier layer consists of EVOH.

8. A device, according to claim 2, wherein the center barrier layer has a thickness of 30-60% of the entire wall.

9. A device, according to claim 2, wherein the center barrier layer has a thickness of 40-50% of the entire wall.

10. A device, according to claim 2, wherein the center barrier layer has a thickness of 45% of the entire wall.

11. A device, according to claim 4, wherein there are breaking points between the closing device and the discharge opening.

12. A device, according to claim 9, wherein the breaking points are notches.

13. A device, according to claim 1, wherein the other layers consist of transparent PET.

14. A device, according to claim 13, wherein the one layer consists of polyamide.

15. A device, according to claim 13, wherein the one layer consists of EVOH.

16. A device, according to claim 1, wherein the one layer consists of polyamide.

17. A device, according to claim 1, wherein the one layer consists of EVOH.

18. A device, according to claim 1, wherein the one layer has a thickness of 30-60% of the entire wall.

19. A device, according to claim 1, wherein the one layer has a thickness of 40-50% of the entire wall.

20. A device, according to claim 1, wherein the one layer has a thickness of 45% of the entire wall.
